(19) United States
(12) Patent Application Publication
(10) Pub. No.: US 2013/0138046 A1
(43) Pub. Date: May 30, 2013

(54) Safe Self-Destruction Syringe with Puncture Proof Protective Sleeve

(71) Applicant: Zhong Feng, Wuxi (CN)

(72) Inventor: Zhong Feng, Wuxi (CN)

(21) Appl. No.: 13/398,780

(22) Filed: Feb. 16, 2012

Related U.S. Application Data
(63) Continuation-in-part of application No. 13/138,613, filed on Nov. 28, 2011.

(51) Int. Cl. A61M 5/32

(52) U.S. Cl. 604/192

(57) ABSTRACT

A syringe includes a tubular syringe body, a plunger device, a needle assembly, a coupling device provided at a fluid passage opening of the syringe body for coupling the needle assembly with the syringe body, a locking member, and a puncture proof protective sleeve selectively and slidably mounted on the syringe body to move from an injection position to a protective position in one-way manner, wherein in the injection position, the puncture proof protective sleeve is slid to receive the syringe body and to expose the needle assembly for injection use, wherein in the protective position, the puncture proof protective sleeve is slid to receive the needle assembly so as to prevent the needle assembly from exposing to an exterior of the puncture proof protective sleeve, wherein the locking member is arranged to lock the puncture proof protective sleeve at the protective position.

### CROSS REFERENCE OF RELATED APPLICATION

This is a Continuation-In-Part application that claims the benefit of priority under 35 U.S.C. §119 to a non-provisional application, application Ser. No. 13/138,613, filed Sep. 9, 2011.

### BACKGROUND OF THE PRESENT INVENTION

1. Field of Invention

The present invention relates to a syringe, and more particularly to a syringe comprising a puncture proof protective sleeve for preventing a user of the syringe to be accidentally hurt by the syringe.

2. Description of Related Arts

Nowadays, almost all medical personnel utilize conventional syringes for injections. A disadvantage of convention syringes is that the needle is not shielded and exposed to an exterior of the syringe so that medical or cleaning staff may easily be hurt by the needles. Because of this problem, syringes having some sorts of safety devices have been developed to avoid used needles to accidentally touch other people. Such safety devices include self-destruction type syringes and syringes with retractable needles. Chinese patent number ZL200410092337.6 and ZL 20040121924.9 disclose syringes with movable caps and represent improvements over the syringes disclosed in U.S. Pat. No. 5,338,310. For these conventional syringes however, the cap cannot be detached from the syringe body. This makes the overall size of the syringes become very bulky, and greatly increases the handling costs of used syringes. Moreover, when the cap is detached from the syringe body, the needle is still exposed to an exterior thereof, and the syringe may easily be reused. Chinese patent number ZL200410092337.6 discloses a syringe in which the protective cap can be detached from the syringe body. This design decreases the handling costs for used syringes, yet it does not prevent the syringe from being easily reused. Chinese patent number ZL2004201219249 and ZL 200410092337.6 discloses another type of syringe in which the various components of the syringes are designed not to follow international standards so as to make reuse of the syringe body much more difficult. However, this type of design makes the hands of the medical staff uncomfortable because of many protrusions formed on an outer side of the syringe body.

### SUMMARY OF THE PRESENT INVENTION

The invention is advantageous in that it provides a syringe comprising a puncture proof protective sleeve for preventing a user of the syringe to be accidentally hurt by the syringe. Moreover, the present invention improves upon Chinese patent number ZL200410092337.6 and ZL200420121924.9, and does not impart uncomfortable feeling to the hands of the relevant medical staff. Moreover, the needle of the present invention is securely retained within a protective sleeve for enhancing a safety feature thereof.

Another advantage of the present invention is to provide a syringe in with a puncture proof protective sleeve, wherein an outer surface of the syringe is kept to be smooth so as to maximize the comfort of the user when he or she uses it for injection on patients.

Another advantage of the present invention is to provide a syringe with a puncture proof protective sleeve, wherein the needle can be securely affixed to a syringe body through a coupling device, wherein this secure connection will not be affected by weather or transportation conditions.

Another advantage of the present invention is to provide a syringe with a puncture proof protective sleeve, wherein when the puncture proof protective sleeve is moved to seal the needle, the protective sleeve is locked and does not detach from the syringe body.

Additional advantages and features of the invention will become apparent from the description which follows, and may be realized by means of the instrumentalities and combinations particular point out in the appended claims.

According to the present invention, the foregoing and other objects and advantages are attained by providing a syringe, comprising:

a tubular syringe body having a fluid passage opening and a predetermined receiving cavity for storing a predetermined amount of liquid and communicated with the fluid passage opening;

a plunger device slidably received in the syringe body for actuating fluid movement in the receiving cavity of the syringe body;

a needle assembly comprising a needle seat and a needle extended from the needle seat;

a coupling device provided at the fluid passage opening of the syringe body for coupling the needle assembly with the syringe body;

a locking member provided at the coupling device; and

a puncture proof protective sleeve having a storage cavity for receiving the syringe body and the needle assembly, wherein the puncture proof protective sleeve is selectively and slidably mounted on the syringe body to move between an injection position and a protective position, wherein in the injection position, the puncture proof protective sleeve is slid to receive the syringe body and to expose the needle assembly for injection use, wherein in the protective position, the puncture proof protective sleeve is slid to receive the needle assembly so as to prevent the needle assembly from exposing to an exterior of the puncture proof protective sleeve, wherein the locking member is arranged to lock the puncture proof protective sleeve at the protective position.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a schematic diagram of a syringe according to a preferred embodiment of the present invention.

FIG. 2 is an exploded side view of a syringe according to the above preferred embodiment of the present invention.

FIG. 3 is a side view of the coupling device of the syringe according to the above preferred embodiment of the present invention.

FIG. 4 is a schematic diagram of the locking member of the syringe according to the above preferred embodiment of the present invention.

FIG. 5 is a schematic diagram of the protective sleeve of the syringe according to the above preferred embodiment of the present invention.

FIG. 6 is a schematic diagram of the syringe according to the above preferred embodiment of the present invention, illustrating that the syringe is used for an injection.

FIG. 7 is a zoom in view (6A) of FIG. 6 according to the above preferred embodiment of the present invention.

FIG. 8 is another schematic diagram of the syringe according to the above preferred embodiment of the present invention.

FIG. 9 is a first alternative mode of the syringe according to the above preferred embodiment of the present invention.

FIG. 10 is a schematic diagram of the plunger shaft and the plunger head according to the above preferred embodiment of the present invention.

FIG. 11 is a second alternative mode of the syringe according to the above preferred embodiment of the present invention.

FIG. 12 is an exploded perspective view of the syringe according to the above preferred embodiment of the present invention.

FIG. 13 is a schematic diagram of the syringe according to the above preferred embodiment of the present invention.

### DETAILED DESCRIPTION OF THE PREFERRED EMBODIMENT

The detailed description of the preferred embodiment of the present invention is as follows:

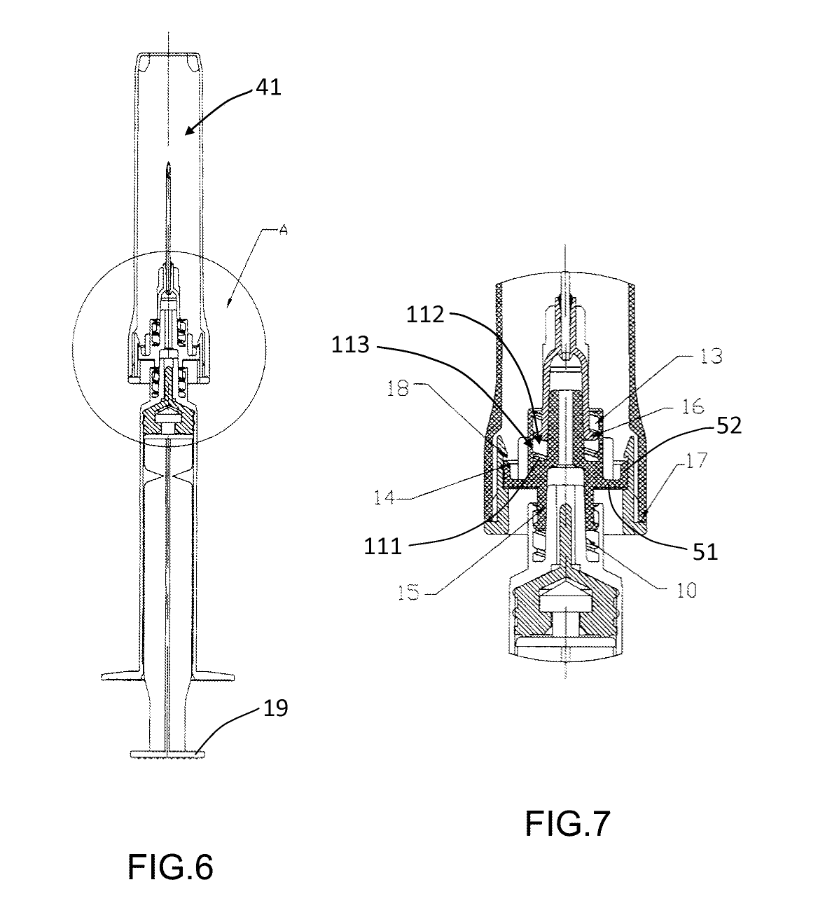

Referring to the drawings, the syringe according to the preferred embodiment of the present invention comprises a needle cap 1, a needle 2, a needle seat 3, a protective sleeve 4, a coupling device 5, a locking member 6, a syringe body 7, a plunger shaft 8, and a plunger head 9. In this preferred embodiment, the coupling device 5 is provided at a top end portion of the protective sleeve 4, while the locking member 6 is received in another end portion of the protective sleeve 4, wherein the locking member 6 is provided at a top end portion of the plunger shaft 8, which are movably received within the syringe body 7. The syringe body 7, the plunger shaft 8, and the plunger head 9 are all operatively received in the protective sleeve 4. The syringe body 7 has a luer taper 10 for connecting with the coupling device 5 through threading mechanism. On the other hand, the coupling device 5 has a circular cross section defining an inner cavity 15, and a plurality of coupling members 16 spacedly extended on the coupling device 5, wherein the coupling members 16 are arranged to couple with the luer taper 10 of the syringe body 7. Furthermore, the needle 2 is extended from the needle seat 3 while the needle cap 1 is detachably affixed on the needle seat 3 to prevent the needle 2 from being exposed to ambient environment. The coupling device 5 further has a connection head 13 wherein the needle seat 3 is detachably attached on the connecting head 13. When the syringe is in clinical use, medical staff may, depending on medical or clinical needs, attach different needle 2 with needle seat 3 an the needle cap 1 onto the coupling device 5. For an injection cycle, a user may first hold a handling sleeve 19 and pulling the plunger shaft 8 backwardly for filling a predetermined medicine into the syringe body 7. When the injection process is finished, the user may slide the protective sleeve 4 toward the needle seat 3 until the locking member 6 is interlocked with the coupling device 5 by means of a resilient flanges 18 (see FIG. 6). It is worth mentioning that the coupling device 5 can be easily detached from the syringe body 7 by rotating the coupling device 5 in a predetermined direction. The syringe disclosed in the present invention improves upon conventional syringes and has a simple structure which facilitates safe and efficient injections. The syringe of the present invention also complies with major international safety standards.

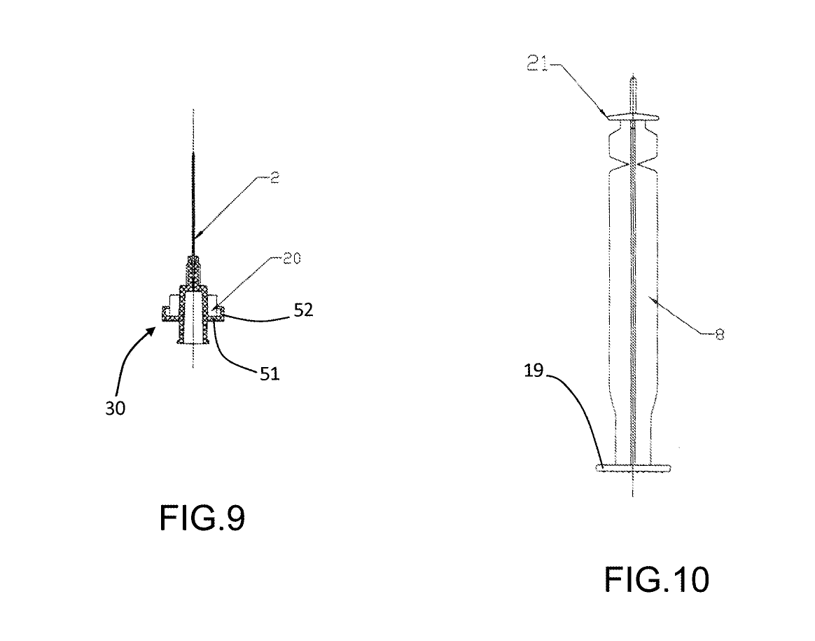

Referring to FIG. 9 of the drawings, an alternative mode of the syringe according to the preferred embodiment of the present invention is illustrated. The alternative mode is similar to the preferred embodiment, except the needle 2 is directly mounted onto the coupling device 5. This arrangement is most suitable for precise injection.

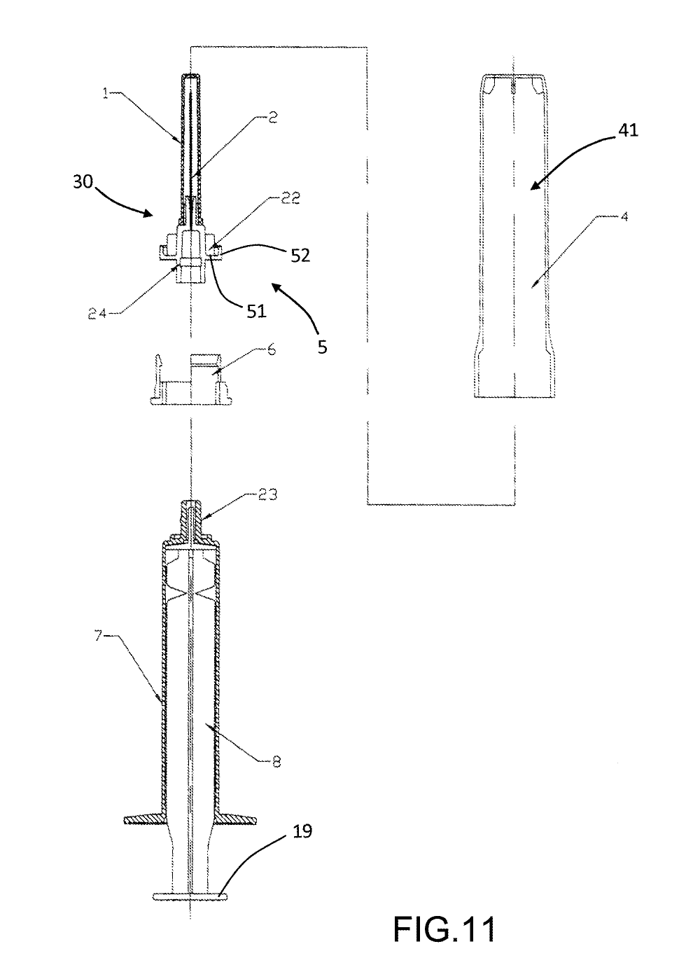

Referring to FIG. 11 of the drawings, a second alternative mode of the preferred embodiment of the present invention is illustrated. The second alternative mode is similar to the preferred embodiment, except the syringe body 7 further comprises an interlocking ring 23 extended from the luer tape 10, wherein the coupling device 5 further has an interlocking slot 24 formed thereon, wherein the interlocking ring 23 is arranged to couple with the interlocking slot 24 so as to enhance the coupling performance between the syringe body 7 and the coupling device 5. Because of this additional feature, the coupling between the syringe body 7 and the coupling device 5 is not affected by transportation or weather conditions so as to produce uniform product quality of the present invention.

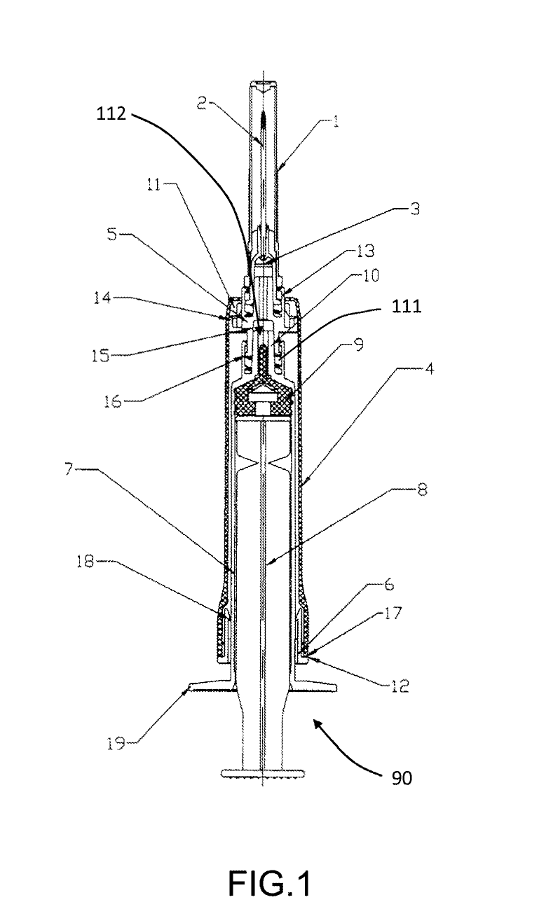

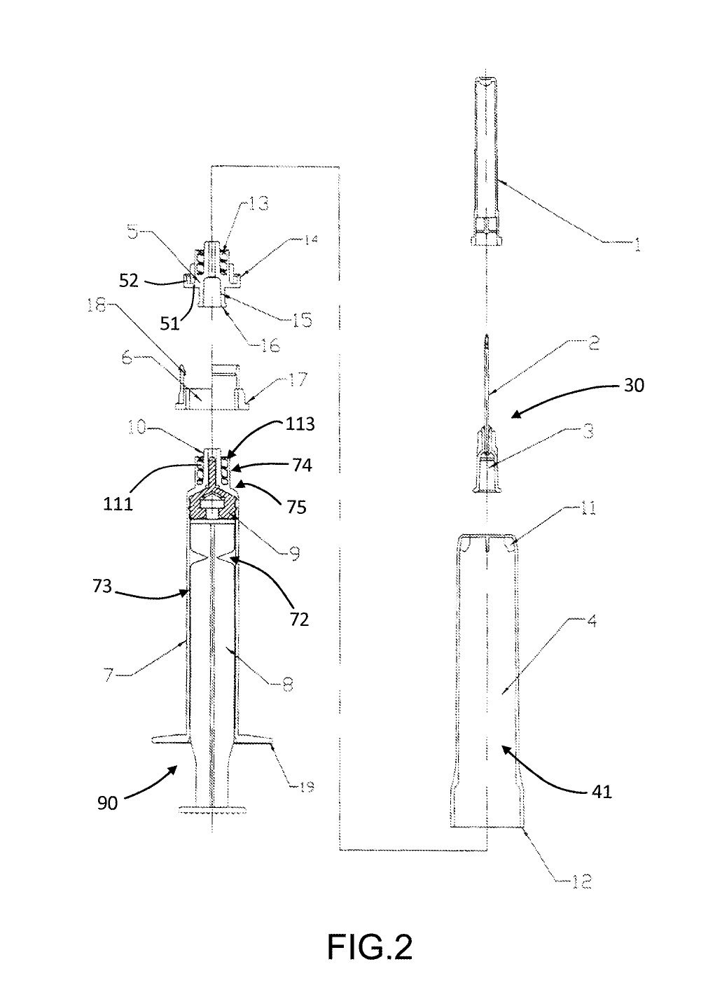

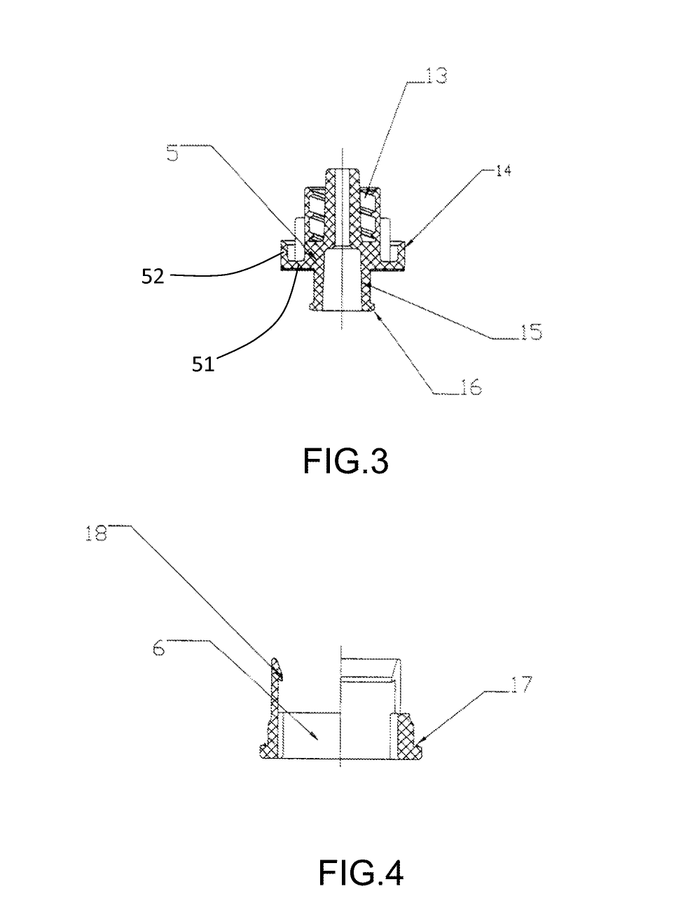

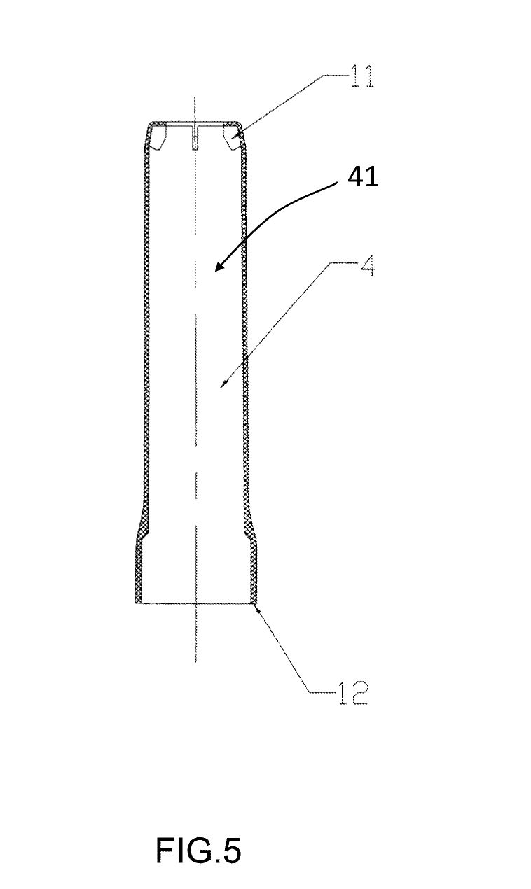

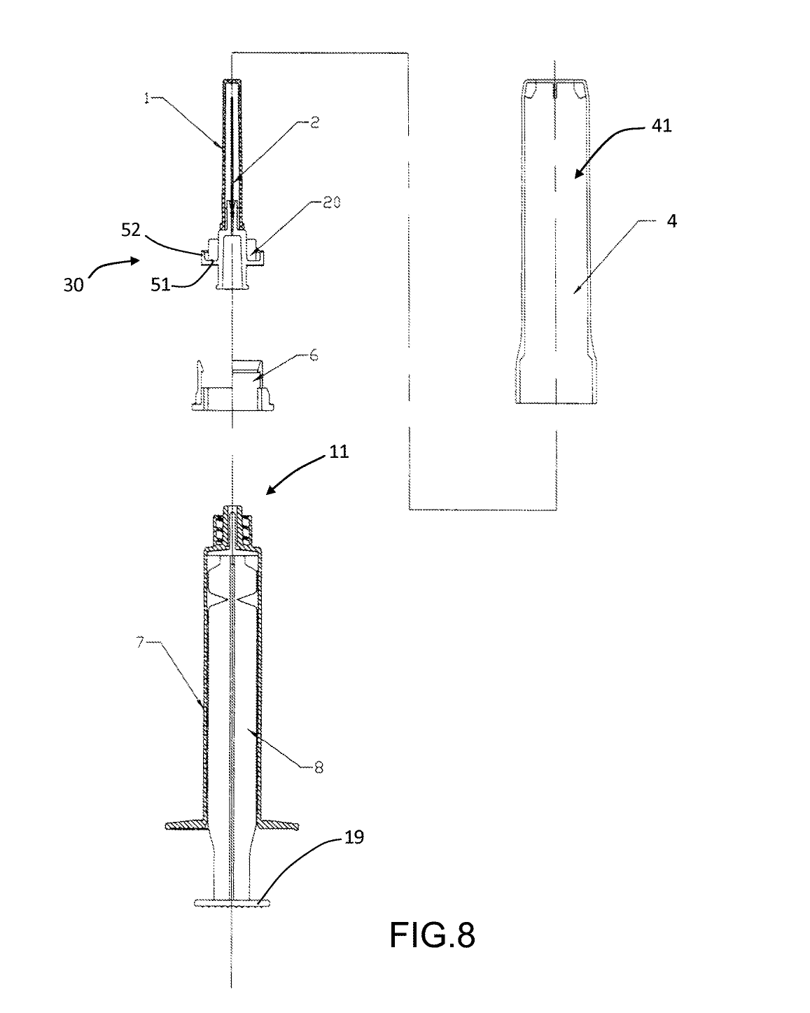

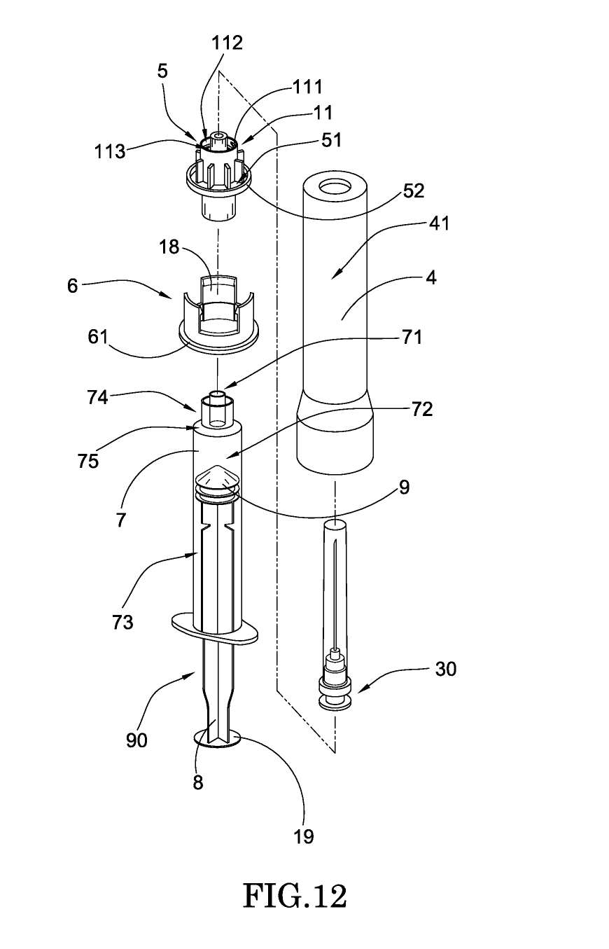

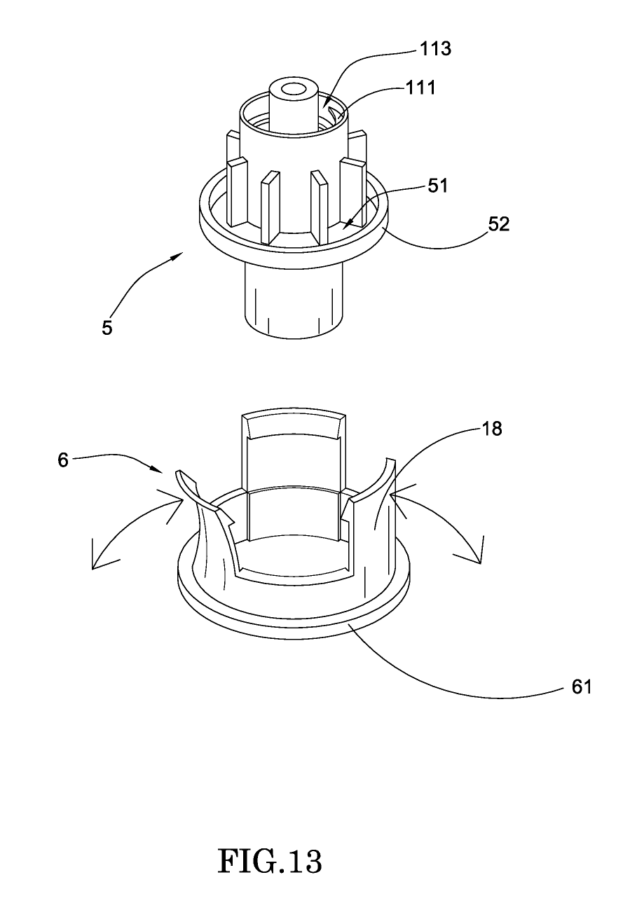

In other words, referring to FIG. 1 to FIG. 13 of the drawings, a syringe according to a preferred embodiment of the present invention is illustrated, in which the syringe comprises a tubular syringe body 7, a plunger device 90, a needle assembly 30, a coupling device 5, a locking member 6, and a puncture proof protective sleeve 4.

The tubular syringe body 7 has a fluid passage opening 71 and a predetermined receiving cavity 72 for storing a predetermined amount of liquid and communicated with the fluid passage opening 71. On the other hand, the plunger device 90 is slidably coupled with the syringe body 7 for actuating fluid movement in the receiving cavity 72 of the syringe body 7. The needle assembly 30 comprising a needle seat 3 and a needle 2 extended from the needle seat 3. The syringe body 7 has a main portion 73 and a contracted opening portion 74 extended from the main portion 73, wherein a diameter of the contracted opening portion 74 is substantially smaller than that of the main portion 73 to define a syringe platform 75 between the main portion 73 and the contracted opening portion 74. It is worth mentioning that the fluid passage opening 71 is formed at the contracted opening portion 74.

The coupling device 5 is securely and non-detachably provided at the fluid passage opening 71 of the syringe body 7 for coupling the needle assembly 30 with the syringe body 7. The locking member 6 is provided at the coupling device 5.

The puncture proof protective sleeve 4 has a storage cavity 41 for receiving the syringe body 7 and the needle assembly 30, wherein the puncture proof protective sleeve 4 is selectively and slidably mounted on the syringe body 7 to move between an injection position and a protective position, wherein in the injection position, the puncture proof protective sleeve 4 is slid to receive the syringe body 7 and to expose the needle assembly 30 for injection use (FIG. 1), wherein in the protective position, the puncture proof protective sleeve 4 is slid to receive the needle assembly 30 so as to prevent the needle assembly 30 from exposing to an exterior of the puncture proof protective sleeve 4, wherein the locking member 6 is arranged to lock the puncture proof protective sleeve 4 at the protective position (FIG. 6).

According to the preferred embodiment of the present invention, the plunger device 90 comprises a plunger shaft 8 and a plunger head 9 formed at one end of the plunger shaft 8, wherein a length of the plunger shaft 8 is larger than a length of the tubular syringe body 7 so that a top end portion of the plunger shaft 8 is exposed out of the receiving cavity 72 for allowing a user to hold thereon. Moreover, the syringe further comprises a handling sleeve 19 transversely extended from a top edge of the tubular sleeve body 7 for accommodating the fingers of the user of the present invention for actuating the movement of the plunger device 90. The plunger head 9 has a diameter slightly smaller than that of the main portion 73 of the syringe body 7 so that the plunger head 9 is capable of longitudinally sliding along the main portion 73.

On the other hand, the luer taper 11 is extended from an outer side of the syringe platform 75 and has a tubular structure to form a luer cavity 112 between an outer side of the contracted opening portion 74 of the syringe body 7 and an inner sidewall 113 of the luer taper 11, wherein the luer taper 11 has a frictional rib 111 extended from the inner sidewall 113 of the luer taper 11 for providing enhanced friction to the needle assembly 30. Accordingly, the needle seat 3 is arranged to detachably couple with the inner sidewall 113 of the luer taper 11 and received in the luer cavity 112.

On the other hand, the puncture proof protective sleeve 4 is also tubular in structure for forming the storage cavity 41, wherein a diameter of a top end is larger than that of a lower end. The storage cavity 41 is arranged to receive the syringe body 7 when the puncture proof protective sleeve 4 is in the injection position. Note that the puncture proof protective sleeve 4 is made of rubber material so that in the ordinary course of event, the puncture proof protective sleeve 4 is puncture proof in the sense that the needle 2 is not capable of hurting people who come to hold or handle the syringe of the present invention.

The locking member 6 comprises a locker ring 61 integrally formed at the top end of the puncture proof protective sleeve 4 within the storage cavity 41, and a plurality of resilient flanges 18 spacedly extended from the locker ring 61, wherein the resilient flanges 18 are arranged to interlock with the coupling device 5 when the puncture proof protective sleeve 4 is in the protective position, so as to restrict an upward movement of the puncture proof protective sleeve 4 once the resilient flanges 18 are interlocked with the coupling device 5.

On the other hand, the coupling device 5 comprises a coupling body 51 and a coupling rim 52 peripherally and downwardly extended from the coupling body 51, wherein the resilient flanges 18 are arranged to interlock with the coupling rim 52 when the puncture proof protective sleeve 4 is at the protective position. More specifically, when the puncture proof protective sleeve 4 is at the protective position, each of the resilient flanges 18, due to its inherent resilient ability, will bias toward the coupling rim 52. Each of the resilient flanges 18 has an interlocking hook 181 inwardly protruded therefrom in such a manner that the interlocking hook 181 is arranged to engage with the coupling rim 52, which prevents the resilient flanges 18 from moving upwardly. In other words, when the puncture proof protective sleeve 4 is at the protective position, the coupling rim 52 restrict an upward movement of the resilient flanges 18 so as to prevent the puncture proof protective sleeve 4 and the attached locking member 6 from moving back to the injection position.

Moreover, in order to prevent the puncture proof protective sleeve 4 from detaching from the syringe body 7, when it is at the protective position, a bottom surface of the locker ring 61 is arranged to bias against a top surface of the coupling body 51 which is securely mounted at the contracted opening portion 74 of the syringe body 7. As a result, a downward movement of the puncture proof protective sleeve 4 is also restricted.

Thus, when the puncture proof protective sleeve 4 is at the protective position, it can no longer go back to the injection position. Since the needle 2 is embedded in the puncture proof protective sleeve 4, the needle 2 and the syringe body 7 cannot be reused and this ensures the maximum hygienic condition of the syringe of the present invention.

The operation of the present invention is as follows: when the puncture proof protective sleeve 4 is at the injection position, a user may attached the needle seat to the contracted opening portion 74 of the syringe body 7 and perform the injection. When the injection has completed, the user may slide the proof protective sleeve 4 to the protective position, and it is retained at that position for maximum protection. The syringe is then readily to be disposable.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A syringe, comprising
a tubular syringe body having a fluid passage opening and a predetermined receiving cavity for storing a predetermined amount of liquid and communicated with said fluid passage opening;
a plunger device slidably coupled with said syringe body for actuating fluid movement in said receiving cavity of said syringe body;
a needle assembly comprising a needle seat and a needle extended from said needle seat;
a coupling device securely provided at said fluid passage opening of said syringe body for coupling said needle assembly with said syringe body;
a locking member; and
a puncture proof protective sleeve having a storage cavity for receiving said syringe body and said needle assembly, wherein said puncture proof protective sleeve is selectively and slidably mounted on said syringe body to move from an injection position to a protective position in one-way manner, wherein in said injection position, said puncture proof protective sleeve is slid to receive said syringe body and to expose said needle assembly for injection use, wherein in said protective position, said puncture proof protective sleeve is slid to receive said needle assembly so as to prevent said needle assembly from exposing to an exterior of said puncture proof protective sleeve, wherein said locking member is arranged to lock said puncture proof protective sleeve at said protective position.

2. The syringe, as recited in claim 1, wherein said syringe body has a main portion and a contracted opening portion extended from said main portion, wherein a diameter of said contracted opening portion is substantially smaller than that of said main portion to define a syringe platform between said main portion and said contracted opening portion, wherein said fluid passage opening is formed at said contracted opening portion.

3. The syringe, as recited in claim 1, wherein said luer taper is extended from an outer side of said syringe platform and has a tubular structure to form a luer cavity between an outer side of said contracted opening portion of said syringe body and an inner sidewall of said luer taper for detachably receiving said needle assembly, wherein said luer taper has a frictional rib extended from said inner sidewall of said luer taper for providing enhanced friction to said needle assembly.

4. The syringe, as recited in claim 2, wherein said luer taper is extended from an outer side of said syringe platform and has a tubular structure to form a luer cavity between an outer side of said contracted opening portion of said syringe body and an inner sidewall of said luer taper for detachably receiving said needle assembly, wherein said luer taper has a frictional rib extended from said inner sidewall of said luer taper for providing enhanced friction to said needle assembly.

5. The syringe, as recited in claim 2, wherein said puncture proof protective sleeve is also tubular in structure for forming said storage cavity, wherein a diameter of a top end is larger than that of a lower end, wherein said puncture proof protective sleeve is made of rubber material so that said puncture proof protective sleeve is puncture proof in a sense that said needle is not capable of hurting a user who comes to hold said syringe.

6. The syringe, as recited in claim 3, wherein said puncture proof protective sleeve is also tubular in structure for forming said storage cavity, wherein a diameter of a top end is larger than that of a lower end, wherein said puncture proof protective sleeve is made of rubber material so that said puncture proof protective sleeve is puncture proof in a sense that said needle is not capable of hurting a user who comes to hold said syringe.

7. The syringe, as recited in claim 4, wherein said puncture proof protective sleeve is also tubular in structure for forming said storage cavity, wherein a diameter of a top end is larger than that of a lower end, wherein said puncture proof protective sleeve is made of rubber material so that said puncture proof protective sleeve is puncture proof in a sense that said needle is not capable of hurting a user who comes to hold said syringe.

8. The syringe, as recited in claim 5, wherein said locking member comprises a locker ring integrally formed at said top end of said puncture proof protective sleeve within said storage cavity, and a plurality of resilient flanges spacedly extended from said locker ring, wherein said resilient flanges are arranged to interlock with said coupling device when said puncture proof protective sleeve is in said protective position, so as to restrict an upward movement of said puncture proof protective sleeve once said resilient flanges are interlocked with said coupling device.

9. The syringe, as recited in claim 6, wherein said locking member comprises a locker ring integrally formed at said top end of said puncture proof protective sleeve within said storage cavity, and a plurality of resilient flanges spacedly extended from said locker ring, wherein said resilient flanges are arranged to interlock with said coupling device when said puncture proof protective sleeve is in said protective position, so as to restrict an upward movement of said puncture proof protective sleeve once said resilient flanges are interlocked with said coupling device.

10. The syringe, as recited in claim 7, wherein said locking member comprises a locker ring integrally formed at said top end of said puncture proof protective sleeve within said storage cavity, and a plurality of resilient flanges spacedly extended from said locker ring, wherein said resilient flanges are arranged to interlock with said coupling device when said puncture proof protective sleeve is in said protective position, so as to restrict an upward movement of said puncture proof protective sleeve once said resilient flanges are interlocked with said coupling device.

11. The syringe, as recited in claim 8, wherein said coupling device comprises a coupling body and a coupling rim peripherally and downwardly extended from said coupling body, wherein said resilient flanges are arranged to interlock with said coupling rim when said puncture proof protective sleeve is at said protective position.

12. The syringe, as recited in claim 9, wherein said coupling device comprises a coupling body and a coupling rim peripherally and downwardly extended from said coupling body, wherein said resilient flanges are arranged to interlock with said coupling rim when said puncture proof protective sleeve is at said protective position.

13. The syringe, as recited in claim 10, wherein said coupling device comprises a coupling body and a coupling rim peripherally and downwardly extended from said coupling body, wherein said resilient flanges are arranged to interlock with said coupling rim when said puncture proof protective sleeve is at said protective position.

14. The syringe, as recited in claim 11, wherein when said puncture proof protective sleeve is at said protective position, each of said resilient flanges, due to an inherent resilient ability, is arranged to bias toward said coupling rim, wherein each of said resilient flanges has an interlocking hook inwardly protruded therefrom in such a manner that said interlocking hook is arranged to engage with said coupling rim, which prevents said resilient flanges from moving upwardly.

15. The syringe, as recited in claim 12, wherein when said puncture proof protective sleeve is at said protective position, each of said resilient flanges, due to an inherent resilient ability, is arranged to bias toward said coupling rim, wherein each of said resilient flanges has an interlocking hook inwardly protruded therefrom in such a manner that said interlocking hook is arranged to engage with said coupling rim, which prevents said resilient flanges from moving upwardly.

16. The syringe, as recited in claim 13, wherein when said puncture proof protective sleeve is at said protective position, each of said resilient flanges, due to an inherent resilient ability, is arranged to bias toward said coupling rim, wherein each of said resilient flanges has an interlocking hook inwardly protruded therefrom in such a manner that said interlocking hook is arranged to engage with said coupling rim, which prevents said resilient flanges from moving upwardly.

17. The syringe, as recited in claim 13, wherein said plunger device comprises a plunger shaft and a plunger head formed at one end of said plunger shaft, wherein a length of said plunger shaft is larger than a length of said tubular syringe body so that a top end portion of said plunger shaft is exposed out of said receiving cavity for allowing a user to hold thereon, wherein said plunger head as a diameter slightly smaller than that of said main portion of said syringe body so that said plunger head is capable of longitudinally sliding along said main portion.

18. The syringe, as recited in claim 14, wherein said plunger device comprises a plunger shaft and a plunger head formed at one end of said plunger shaft, wherein a length of said plunger shaft is larger than a length of said tubular syringe body so that a top end portion of said plunger shaft is exposed out of said receiving cavity for allowing a user to hold thereon, wherein said plunger head as a diameter slightly smaller than that of said main portion of said syringe body so that said plunger head is capable of longitudinally sliding along said main portion.

19. The syringe, as recited in claim 15, wherein said plunger device comprises a plunger shaft and a plunger head formed at one end of said plunger shaft, wherein a length of said plunger shaft is larger than a length of said tubular syringe body so that a top end portion of said plunger shaft is exposed out of said receiving cavity for allowing a user to hold thereon, wherein said plunger head as a diameter slightly smaller than that of said main portion of said syringe body so that said plunger head is capable of longitudinally sliding along said main portion.

20. The syringe, as recited in claim 16, wherein said plunger device comprises a plunger shaft and a plunger head formed at one end of said plunger shaft, wherein a length of said plunger shaft is larger than a length of said tubular syringe body so that a top end portion of said plunger shaft is exposed out of said receiving cavity for allowing a user to hold thereon, wherein said plunger head as a diameter slightly smaller than that of said main portion of said syringe body so that said plunger head is capable of longitudinally sliding along said main portion.
